(19) United States
(12) Patent Application Publication
(10) Pub. No.: US 2023/0093124 A1
(43) Pub. Date: Mar. 23, 2023

(54) Salts of Nintedanib and Crystalline Forms Thereof

(71) Applicant: Apotex Inc., Toronto (CA)

(72) Inventors: Fabio E. S. Souza, Brantford (CA); Alexander J. Stirk, Brantford (CA); Avedis Karadeolian, Brantford (CA); Fatemeh Mohammadpourmir, Brantford (CA); Allan W. Rey, Brantford (CA)

(21) Appl. No.: 17/947,287

(22) Filed: Sep. 19, 2022

(30) Foreign Application Priority Data
Sep. 20, 2021 (CA) 3131364

(51) Int. Cl. C07D 209/34

(52) U.S. Cl. CPC C07D 209/34

(57) ABSTRACT

Provided are novel salts of nintedanib and crystalline forms thereof. Specific salts of nintedanib provided by the present invention include glutarate, hippurate, levulinate, acesulfamate, diacesulfamate, and saccharinate. Also provided are pharmaceutical compositions including the nintedanib salts and crystalline forms thereof, and the use of these salts in the treatment or prevention of fibrotic diseases selected from the group including idiopathic pulmonary fibrosis, chronic fibrosing interstitial lung disease with a progressive phenotype, and systemic sclerosis-associated interstitial lung disease.

### CROSS-REFERENCE TO RELATED APPLICATION

This application claims priority to Canadian Patent Application No. 3131364 filed Sep. 20, 2021, the disclosure of which is hereby incorporated by reference in its entirety.

### BACKGROUND OF THE INVENTION

### Field of the Invention

The present invention is directed to novel salts of nintedanib, crystalline forms, pharmaceutical compositions containing these salts, and their use for the treatment or prevention of fibrotic diseases.

### Description of Related Art

Nintedanib (1), or (3Z)-2,3-dihydro-3-[[[4-[methyl[(4-methyl-1-piperazinyl)acetyl]amino]phenyl]amino]phenylmethylene]-2-oxo-1H-indole-6-carboxylic acid methyl ester, in the form of its ethanesulfonate (esylate) salt (1:1), is the active pharmaceutical ingredient (API) in the branded pharmaceutical OFEV®, a prescription medication for use in the treatment of certain fibrotic diseases. According to the product label, “OFEV® capsules for oral administration are available in 2 dose strengths containing 100 mg or 150 mg of nintedanib (equivalent to 120.40 mg or 180.60 mg nintedanib ethanesulfonate, respectively). The inactive ingredients of OFEV® are the following: Fill Material: triglycerides, hard fat, lecithin. Capsule Shell: gelatin, glycerol, titanium dioxide, red ferric oxide, yellow ferric oxide, black ink.”

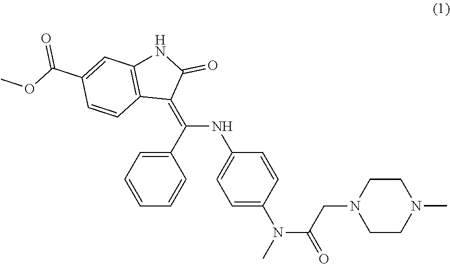

Nintedanib salts and crystalline forms thereof are reported in, for example, WO 2004/013099 A1, WO 2007/141283 A1, WO 2012/068441 A1, WO 2016/146020 A1, WO 2017/144029 A1, WO 2017/198202 A1, WO 2019/241504 A1, and WO 2020/041631 A1.

According to the European CHMP Assessment Report for OFEV® (EMEA/H/C/003821/0000), nintedanib esylate has poor solubility in neutral aqueous conditions and low bioavailability in humans. The OFEV® drug product is provided as a soft gelatin capsule, wherein the nintedanib esylate is suspended in a lipophilic fill mixture of triglycerides, hard fat and lecithin. According to WO 2009/147212 A1, which likewise describes a suspension formulation of nintedanib esylate in the form of a soft gelatin capsule, avoidance of physical stability issues, such as re-crystallization or particle-growth, requires the active substance to be either completely insoluble or dissolved in the carrier. Given the characterization of the OFEV® drug product filling as a suspension, insolubility of the nintedanib esylate in the lipophilic fill mixture could be an important factor in supporting physical stability. WO 2009/147212 A1 further describes the effect of water content on the chemical stability of the nintedanib esylate, which can reportedly undergo hydrolysis in the presence of water.

The solubility of individual salt and crystalline forms of a drug substance in an aqueous environment is an important aspect of their relative bioavailability, since the manner in which the salt or crystalline form dissolves can correspond to the amount of the drug substance that is available to be absorbed into the body to provide the intended therapeutic effect. One measure of solubility is intrinsic dissolution rate (IDR), which is defined as the dissolution rate of a substance under constant surface area conditions. For low solubility substances, higher IDR values can correlate with higher bioavailability following administration. However, if the goal is to establish bioequivalence to an existing form of a drug under investigation, substances with similar IDR values to the known form are preferred. Alternatively, for the development of extended or sustained release products, forms exhibiting lower IDR values are often preferable since they can provide slower dissolution of the drug independent of the excipients used in the formulation.

The nintedanib salt used in the OFEV® drug product is an ethanesulfonate salt. Use of sulfonic acid salts of APIs can trigger concerns from drug regulatory agencies related to the possible presence of potential mutagenic sulfonic acid ester impurities arising from, for example, reaction between the sulfonic acid and an alcohol that may be present. Use of sulfonic acid salts of APIs can even result in a regulatory requirement to demonstrate detection and control of potential alkyl ethanesulfonates at very low (ppm) levels, which can be burdensome. Of note, the ethanesulfonate salt reported in WO 2004/013099 A1 is prepared in the presence of methanol solvent and the OFEV® drug product contains glycerol in the capsule shell.

Different crystalline and/or salt forms of the same compound may have different crystal packing, thermodynamic, spectroscopic, kinetic, surface and mechanical properties. For example, different salts and/or crystalline forms may have different stability properties such that a particular form may be less sensitive to heat, relative humidity (RH) and/or light. Different salts and/or crystalline forms of a compound may also be more susceptible to moisture uptake, resulting in a potential alteration of the chemical stability, such as when the compound is susceptible to hydrolysis. Different salts may exist in more than one crystalline form, which can cause complexity in ensuring the stability of a desired crystalline form in a drug product such as a suspension formulation.

For example, a particular salt and/or crystalline form may provide more favourable compressibility and/or density properties, thereby providing more desirable characteristics for formulation and/or product manufacturing. Differences in stability between salts and/or crystalline forms of a drug may result from changes in chemical reactivity, such as differential oxidation. The melting point of a particular salt and/or crystalline form, particularly a low melting point, can contribute to issues during processing, which impact on both flow and compressibility performance. Particular salts and/or crystalline forms may also have different solubilities, in both lipophilic and aqueous environments, with implications related to formulation options and pharmacokinetics.

Although general approaches to salt and crystalline form screening of active pharmaceutical ingredients are known, it is well established that the prediction of whether any given compound will exhibit polymorphism is not possible. Accordingly, it is not possible to extend generalities to the number and kinds of crystalline forms that can exist for nintedanib salts, or to what methods will be suitable for the preparation of any given form. Furthermore, prediction of the properties of any unknown salts and/or crystalline forms, and how they will differ from other crystalline forms or salts of the same compound, remains elusive (Joel Bernstein, Polymorphism in Molecular Crystals, Oxford University Press, New York, 2002, page 9).

Given the solubility, stability, bioavailability, and regulatory considerations that exist for nintedanib esylate, there is a need for novel salts of nintedanib and crystalline forms thereof for use in improved drug products, and commercially amenable processes for their manufacture.

### SUMMARY OF THE INVENTION

The nintedanib salts and crystalline forms of the present invention comprise organic acids having an established safety record. Embodiments of the present invention incorporate first class acids according to a notable reference book on the pharmaceutical acceptability of salts: P. Heinrich Stahl, Camille G. Wermuth (Eds.), Handbook of Pharmaceutical Salts Properties, Selection, and Use; 2002. First class acids are classified by Stahl et al. as those that afford physiologically ubiquitous ions or metabolites in biochemical pathways, supporting their unrestricted use in pharmaceuticals. In other embodiments, salts of nintedanib are provided with acids that are used in the food industry, including acesulfame, levulinic acid, and saccharin, with the expectation that these acids can safely be used in materials intended for use in the preparation of pharmaceutical compositions intended for administration to humans. Further, embodiments of the invention exhibit form stability at high temperature and high humidity.

In addition, the processes for the manufacture of the nintedanib salts and crystalline forms of the present invention are efficient and industrially compatible.

Accordingly, in a first aspect of the present invention, there is provided a glutarate salt of nintedanib. In a preferred embodiment of the first aspect, the molar ratio of nintedanib to glutaric acid is approximately 1:1. In a more preferred embodiment of the first aspect, the salt is characterized by a PXRD diffractogram comprising peaks, expressed in degrees 2θ (±0.2°), at 10.1° and 12.9°. More preferably, the salt of the first aspect is characterized by a PXRD diffractogram further comprising at least four peaks, expressed in degrees 2θ (±0.2°), selected from the group consisting of: 11.0°, 15.1°, 17.4°, 18.0°, 19.5°, and 20.2°. In a further preferred embodiment of the first aspect, the PXRD diffractogram further comprises peaks, expressed in degrees 2θ (±0.2°), at 11.0°, 15.1°, 17.4°, 18.0°, 19.5°, and 20.2°. Preferably, the salt of the first aspect of the invention provides a PXRD diffractogram comprising peaks in substantially the same positions (±0.2° 28) as those shown in FIG. 1.

In a second aspect of the present invention, there is provided a hippurate salt of nintedanib. In a preferred embodiment of the second aspect, the molar ratio of nintedanib to hippuric acid is approximately 1:1. In a more preferred embodiment of the second aspect, the salt is characterized by a PXRD diffractogram comprising peaks, expressed in degrees 2θ (±0.2°), at 9.1° and 9.8°. More preferably, the salt of the second aspect is characterized by a PXRD diffractogram further comprising at least four peaks, expressed in degrees 2θ (±0.2°), selected from the group consisting of: 10.1°, 10.6°, 13.0°, 13.3°, 14.6°, and 20.9°. In a further preferred embodiment of the second aspect, the PXRD diffractogram further comprises peaks, expressed in degrees 2θ (±0.2°), at 10.1°, 10.6°, 13.0°, 13.3°, 14.6°, and 20.9°. Preferably, the salt of the second aspect of the invention provides a PXRD diffractogram comprising peaks in substantially the same positions (±0.2° 28) as those shown in FIG. 2.

In a third aspect of the present invention, there is provided a levulinate salt of nintedanib. In a preferred embodiment of the third aspect, the molar ratio of nintedanib to levulinic acid is approximately 1:1. In a more preferred embodiment of the third aspect, the salt is characterized by a PXRD diffractogram comprising peaks, expressed in degrees 2θ (±0.2°), at 8.8° and 10.9°. More preferably, the salt of the third aspect is characterized by a PXRD diffractogram further comprising at least four peaks, expressed in degrees 2θ (±0.2°), selected from the group consisting of: 8.1°, 11.6°, 12.1°, 16.3°, 17.6°, and 20.5°. In a further preferred embodiment of the third aspect, the PXRD diffractogram further comprises peaks, expressed in degrees 2θ (±0.2°), at 8.1°, 11.6°, 12.1°, 16.3°, 17.6°, and 20.5°. Preferably, the salt of the third aspect of the invention provides a PXRD diffractogram comprising peaks in substantially the same positions (±0.2° 28) as those shown in FIG. 3.

In a fourth aspect of the present invention, there is provided an acesulfamate salt of nintedanib. In a first preferred embodiment of the fourth aspect, the molar ratio of nintedanib to acesulfame is approximately 1:1. In a more preferred embodiment, the nintedanib acesulfamate salt is characterized by a PXRD diffractogram comprising peaks, expressed in degrees 2θ (±0.2°), at 12.9° and 14.6°. More preferably, the nintedanib acesulfamate salt is characterized by a PXRD diffractogram further comprising at least four peaks, expressed in degrees 2θ (±0.2°), selected from the group consisting of: 10.9°, 12.3°, 17.2°, 18.1°, 20.9°, and 21.4°. In a further preferred embodiment, the PXRD diffractogram of the nintedanib acesulfamate salt further comprises peaks, expressed in degrees 2θ (±0.2°), at 10.9°, 12.3°, 17.2°, 18.1°, 20.9°, and 21.4°. Preferably, the nintedanib acesulfamate salt provides a PXRD diffractogram comprising peaks in substantially the same positions (±0.2° 28) as those shown in FIG. 4.

In a second preferred embodiment of the fourth aspect, the molar ratio of nintedanib to acesulfame is approximately 1:2. In a more preferred embodiment, the nintedanib diacesulfamate salt is characterized by a PXRD diffractogram comprising peaks, expressed in degrees 2θ (±0.2°), at 6.4° and 7.9°. More preferably, the nintedanib diacesulfamate salt is characterized by a PXRD diffractogram further comprising at least four peaks, expressed in degrees 2θ (±0.2°), selected from the group consisting of: 12.0°, 12.8°, 13.3°, 14.6°, 17.1°, and 19.3°. In a further preferred embodiment, the PXRD diffractogram of the nintedanib diacesulfamate salt further comprises peaks, expressed in degrees 2θ (±0.2°), at 12.0°, 12.8°, 13.3°, 14.6°, 17.1°, and 19.3°. Preferably, the nintedanib diacesulfamate salt provides a PXRD diffractogram comprising peaks in substantially the same positions (±0.2° 2θ) as those shown in FIG. 5.

In a fifth aspect of the present invention, there is provided a saccharinate salt of nintedanib, wherein the molar ratio of nintedanib to saccharin is approximately 1:1 and the salt is characterized by a PXRD diffractogram comprising peaks, expressed in degrees 2θ (±0.2°), at 8.1° and 15.7°. More preferably, the salt of the fifth aspect is characterized by a PXRD diffractogram further comprising at least four peaks, expressed in degrees 2θ (±0.2°), selected from the group consisting of: 7.1°, 8.8°, 10.5°, 12.3°, 17.9°, and 20.5°. In a further preferred embodiment of the fifth aspect, the PXRD diffractogram further comprises peaks, expressed in degrees 2θ (±0.2°), at 7.1°, 8.8°, 10.5°, 12.3°, 17.9°, and 20.5°. Preferably, the salt of the fifth aspect of the invention provides a PXRD diffractogram comprising peaks in substantially the same positions (±0.2° 28) as those shown in FIG. 6.

In a sixth aspect of the present invention, there is provided a pharmaceutical composition comprising a salt of nintedanib according to the first, second, third, fourth or fifth aspects of the invention, and one or more pharmaceutically acceptable excipients. Preferably, the pharmaceutical composition is in the form of a capsule or a tablet. Most preferably, the pharmaceutical composition is a soft gelatin capsule. Preferably, the pharmaceutical composition of the sixth aspect comprises an amount of the nintedanib salt of the first, second, third, fourth or fifth aspect that is equivalent to 100 or 150 mg of nintedanib free base.

In a seventh aspect of the present invention, there is provided the use of a salt of nintedanib according to the first, second, third, fourth or fifth aspects of the invention, or the pharmaceutical compositions of the sixth aspect of the invention, in the treatment of a fibrotic disease. In a preferred embodiment of the seventh aspect, the fibrotic disease is selected from the group consisting of idiopathic pulmonary fibrosis, chronic fibrosing interstitial lung diseases with a progressive phenotype, and systemic sclerosis-associated interstitial lung disease.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention are described, by way of example only, with reference to the attached Figures.

FIG. 1 is a representative PXRD diffractogram of nintedanib glutarate Form APO-I as prepared in Example 1.

FIG. 2 is a representative PXRD diffractogram of nintedanib hippurate Form APO-I as prepared in Example 2.

FIG. 3 is a representative PXRD diffractogram of nintedanib levulinate Form APO-I as prepared in Example 3.

FIG. 4 is a representative PXRD diffractogram of nintedanib acesulfamate Form APO-I as prepared in Example 4.

FIG. 5 is a representative PXRD diffractogram of nintedanib diacesulfamate Form APO-I as prepared in Example 5.

FIG. 6 is a representative PXRD diffractogram of nintedanib saccharinate Form APO-I as prepared in Example 6.

FIG. 7 is a representative PXRD diffractogram of nintedanib saccharinate Form APO-II as prepared in Example 6.

### DESCRIPTION OF THE INVENTION

The present invention provides nintedanib salts and crystalline forms thereof which comprise organic acids having an established safety record. Glutaric acid and hippuric acid are first class acids according to a reference book on the pharmaceutical acceptability of salts: P. Heinrich Stahl, Camille G. Wermuth (Eds.), Handbook of Pharmaceutical Salts Properties, Selection, and Use; 2002. First class acids are classified by Stahl et al. as those that afford physiologically ubiquitous ions or metabolites in biochemical pathways. Glutaric and hippuric acids are water-soluble metabolites which occur naturally in the human body. Acesulfame, levulinic acid, and saccharin are acids used as food additives and are also included in both the U.S. Food & Drug Administration's (FDA's) Substances Added to Food inventory (formerly Everything Added to Food in the United States (EAFUS)) list and the Inactive Ingredient Database (IID). The Substances Added to Food inventory contains approximately 4,000 substances, and includes information on food additives, colour additives, Generally Recognized As Safe (GRAS) substances, and prior-sanctioned substances. The IID list provides information on inactive ingredients present in FDA-approved drug products. Once an inactive ingredient has appeared in an approved drug product, the inactive ingredient is not considered new, and may require a less extensive review the next time it is included in a new drug product.

The present invention provides salts of nintedanib and crystalline forms thereof providing improved properties over known salts of nintedanib. Depending on the specific salts and crystalline form of the invention used, properties that differ between the invention and known forms of nintedanib include the following: packing properties such as molar volume, density and hygroscopicity; thermodynamic properties such as melting point and solubility; kinetic properties such as dissolution rate and chemical/polymorphic stability; surface properties such as crystal habit; and/or mechanical properties such as hardness, tensile strength, cohesiveness, compactibility, tableting, handling, flow, and blending.

Additionally, the processes for the manufacture of the nintedanib salts and crystalline forms of the present invention are efficient and industrially compatible, with embodiments using Class 3 solvents established by the ICH (International Council for Harmonization of Technical Requirements for Pharmaceuticals for Human Use) as having low toxicity.

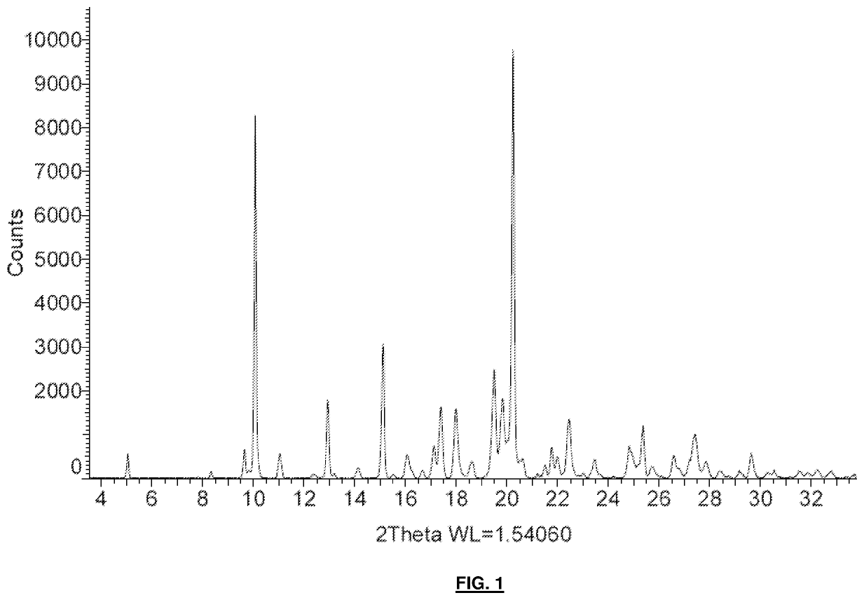

Depending on the manner in which the crystalline forms of the present invention are prepared, and the methodology and instrument used for PXRD analysis, the intensity of a given peak observed in a PXRD diffractogram of the crystalline form may vary when compared to the same peak in the representative PXRD diffractograms provided in FIGS. 1 to 7. Thus, differences in relative peak intensities between peaks in a PXRD diffractogram for a given crystalline form may be observed when compared to the relative peak intensities of the peaks in the representative PXRD diffractograms of FIGS. 1 to 7. Any such differences may be due, in part, to the preferred orientation of the sample and its deviation from the ideal random sample orientation, the preparation of the sample for analysis, and the methodology applied for the analysis. Such variations are known and understood by a person of skill in the art, and any such variations do not depart from the invention disclosed herein.

In addition to the differences in relative peak intensities that may be observed in comparison to the representative PXRD diffractograms provided in FIGS. 1 to 7, it is understood that individual peak positions may vary between ±0.2° 2θ from the values observed in the representative PXRD diffractograms provided in FIGS. 1 to 7 for the crystalline forms of the invention, or listed in Tables 1 to 6. Such variations are known and understood by a person of skill in the art, and any such variations do not depart from the invention disclosed herein.

Further, depending on the instrument used for X-ray analysis and its calibration, uniform offsets in the peak position of each peak in a PXRD diffractogram of greater that 0.2° 2θ may be observed when compared to the representative PXRD diffractograms provided in FIGS. 1 to 7. Thus, the PXRD diffractogram of the crystalline forms of the present invention may, in some circumstances, display the same relative peak positions as observed in the representative PXRD diffractograms provided in FIGS. 1 to 7, with the exception that each peak is offset in the same direction, and by approximately the same amount, such that the overall PXRD diffractogram is substantially the same in appearance as the PXRD diffractograms of FIGS. 1 to 7, with the exception of the uniform offset in peak positions. The observation of any such uniform peak shift in a PXRD diffractogram does not depart from the invention disclosed herein given that the relative peak positions of the individual peaks within the PXRD diffractogram remain consistent with the relative peak positions observed in the PXRD diffractograms of FIGS. 1 to 7.

As used herein, the term ‘crystalline form’ refers to a nintedanib salt of fixed composition with a particular arrangement of components in its crystal lattice, and which may be identified by physical characterization methods such as PXRD. As used herein, the term crystalline form is intended to include single-component and multiple-component crystalline forms of a nintedanib salt. Single-component forms of a nintedanib salt consist solely of nintedanib and the counterion in the repeating unit of the crystal lattice. Multiple-component forms of a nintedanib salt include cocrystals and solvates of a nintedanib salt wherein a coformer or solvent is also incorporated into the crystal lattice.

As used herein, the term “room temperature” refers to a temperature in the range of 20° C. to 25° C.

Unless defined otherwise herein, the term “approximately”, when used in reference to molar ratios, allows for a variance of plus or minus 10%.

When describing the embodiments of the present invention there may be a common variance to a given temperature or time that would be understood or expected by the person skilled in the art to provide substantially the same result. For example, when reference is made to a particular temperature, it is to be understood by the person skilled in the art that there is an allowable variance of ±5° C. associated with that temperature. When reference is made to a particular time, it is to be understood that there is an allowable variance of ±10 minutes when the time is one or two hours, and ±1 hour when longer periods of time are referenced.

In one embodiment of the present invention, there is provided a new salt of nintedanib, nintedanib glutarate Form APO-I, wherein the molar ratio of nintedanib to glutaric acid is approximately 1:1.

Nintedanib glutarate Form APO-I can be characterized by a PXRD diffractogram comprising, among other peaks, characteristic peaks, expressed in degrees 2θ (±0.2°), at 10.1° and 12.9°. Preferably, the PXRD diffractogram further comprises at least four peaks, expressed in degrees 2θ (±0.2°), selected from the group consisting of 11.0°, 15.1°, 17.4°, 18.0°, 19.5°, and 20.2°. More preferably, the PXRD diffractogram further comprises peaks, expressed in degrees 2θ (±0.2°), at 11.0°, 15.1°, 17.4°, 18.0°, 19.5°, and 20.2°.

An illustrative PXRD diffractogram of nintedanib glutarate Form APO-I, as prepared in Example 1, is shown in FIG. 1. A peak listing, comprising representative peaks from the PXRD diffractogram in FIG. 1, and their relative intensities, is provided in Table 1. Although illustrative of the PXRD diffractogram that is provided for the nintedanib glutarate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 1
Relative peak intensities of nintedanib
glutarate Form APO-I from FIG. 1

| Angle (2θ) | Relative intensity (%) |
|---|---|
| 5.06 | 5.6 |
| 9.65 | 6.7 |
| 10.07 | 84.2 |
| 11.04 | 5.7 |
| 12.94 | 18.3 |
| 15.11 | 31.2 |
| 16.06 | 5.6 |
| 17.40 | 16.7 |
| 17.98 | 16.2 |
| 19.50 | 25.4 |
| 19.82 | 18.7 |
| 20.24 | 100.0 |
| 21.77 | 7.3 |
| 22.44 | 13.8 |

As described in Example 1, nintedanib glutarate Form APO-I can be prepared by maintaining a suspension of nintedanib and glutaric acid, preferably approximately equimolar amounts, in a mixture of isopropanol and water at an elevated temperature, preferably between about 40° C. and about 60° C. for a suitable time, preferably between about 3 hours and about 10 hours. Preferably, the suspension is allowed to cool to room temperature prior to isolation and drying, if necessary, preferably drying in vacuo and at room temperature.

In another embodiment of the present invention, there is provided a new salt of nintedanib, nintendanib hippurate Form APO-I, wherein the molar ratio of nintedanib to hippuric acid is approximately 1:1.

Nintedanib hippurate Form APO-I can be characterized by a PXRD diffractogram comprising, among other peaks, characteristic peaks, expressed in degrees 2θ (±0.2°), at 9.1° and 9.8°. Preferably, the PXRD diffractogram further comprises at least four peaks, expressed in degrees 2θ (±0.2°), selected from the group consisting of 10.1°, 10.6°, 13.0°, 13.3°, 14.6°, and 20.9°. More preferably, the PXRD diffractogram further comprises peaks, expressed in degrees 2θ (±0.2°), at 10.1°, 10.6°, 13.0°, 13.3°, 14.6°, and 20.9°.

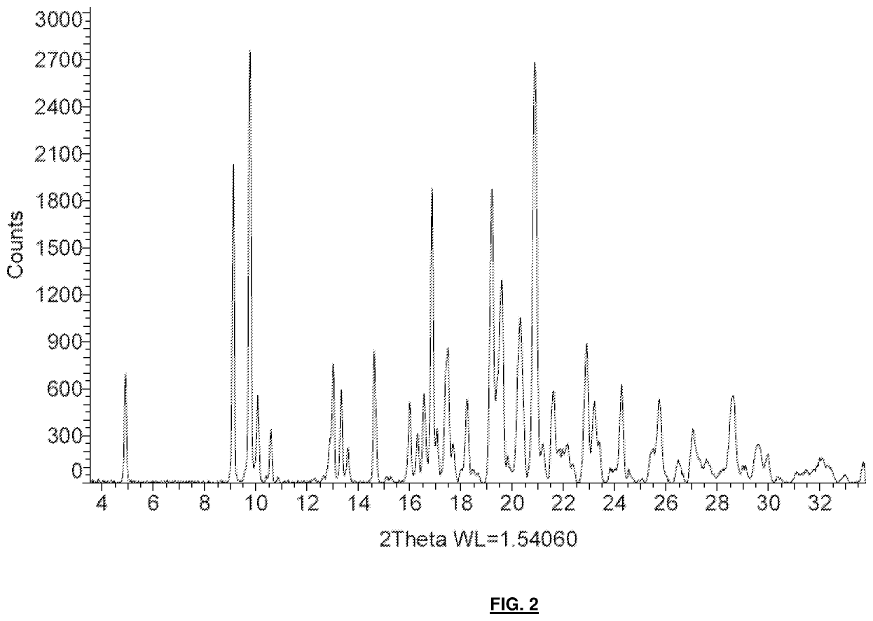

An illustrative PXRD diffractogram of nintedanib hippurate Form APO-I, as prepared in Example 2, is shown in FIG. 2. A peak listing, comprising representative peaks from the PXRD diffractogram in FIG. 2, and their relative intensities, is provided in Table 2. Although illustrative of the PXRD diffractogram that is provided for the nintedanib hippurate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 2
Relative peak intensities of nintedanib
hippurate Form APO-I from FIG. 2

| Angle (2θ) | Relative intensity (%) |
|---|---|
| 4.91 | 25.0 |
| 9.13 | 73.4 |
| 9.77 | 100.0 |
| 10.08 | 20.3 |
| 10.58 | 12.2 |
| 13.02 | 27.5 |
| 13.33 | 21.5 |
| 14.61 | 30.6 |
| 16.00 | 18.6 |
| 16.31 | 11.3 |
| 16.56 | 20.6 |
| 16.87 | 68.3 |
| 17.49 | 31.1 |
| 18.25 | 19.3 |
| 19.21 | 67.8 |
| 19.59 | 47.0 |
| 20.31 | 38.2 |
| 20.89 | 97.1 |
| 22.90 | 32.1 |

As described in Example 2, nintedanib Hippurate Form APO-I can be prepared by maintaining a suspension of nintedanib and hippuric acid, preferably approximately equimolar amounts, in isopropanol at an elevated temperature, preferably between about 40° C. and about 60° C. for a suitable time, preferably between about 3 hours and about 10 hours. Preferably, the suspension is allowed to cool to room temperature prior to isolation and drying, if necessary, preferably drying in vacuo and at room temperature.

In another embodiment of the present invention, there is provided a new salt of nintedanib, nintedanib levulinate Form APO-I, wherein the molar ratio of nintedanib to levulinic acid is approximately 1:1.

Nintedanib levulinate Form APO-I can be characterized by a PXRD diffractogram comprising, among other peaks, characteristic peaks, expressed in degrees 2θ (±0.2°), at 8.8° and 10.9°. Preferably, the PXRD diffractogram further comprises at least four peaks, expressed in degrees 2θ (±0.2°), selected from the group consisting of 8.1°, 11.6°, 12.1°, 16.3°, 17.6°, and 20.5°. More preferably, the PXRD diffractogram further comprises peaks, expressed in degrees 2θ (±0.2°), at 8.1°, 11.6°, 12.1°, 16.3°, 17.6°, and 20.5°.

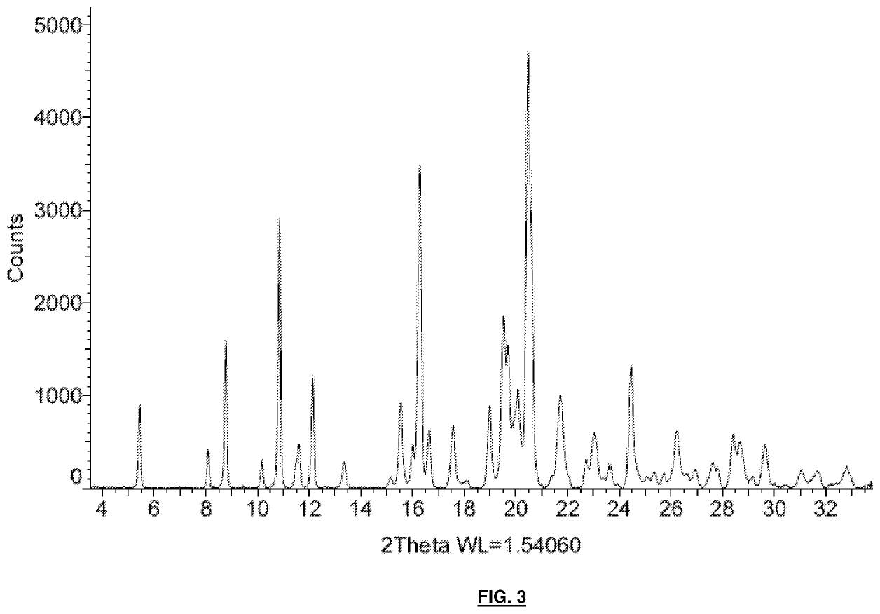

An illustrative PXRD diffractogram of nintedanib levulinate Form APO-I, as prepared in Example 3, is shown in FIG. 3. A peak listing, comprising representative peaks from the PXRD diffractogram in FIG. 3, and their relative intensities, is provided in Table 3. Although illustrative of the PXRD diffractogram that is provided for the nintedanib levulinate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 3
Relative peak intensities of nintedanib
levulinate Form APO-I from FIG. 3

| Angle (2θ) | Relative intensity (%) |
|---|---|
| 5.45 | 18.9 |
| 8.10 | 8.7 |
| 8.78 | 34.0 |
| 10.18 | 6.4 |
| 10.86 | 61.7 |
| 11.60 | 10.1 |
| 12.15 | 25.6 |
| 13.36 | 6.1 |
| 15.56 | 19.5 |
| 16.29 | 73.9 |
| 16.66 | 13.3 |
| 17.58 | 14.4 |
| 18.99 | 18.9 |
| 20.08 | 22.5 |
| 20.48 | 100.0 |
| 21.73 | 21.2 |

As described in Example 3, nintedanib levulinate Form APO-I can be prepared by maintaining a suspension of nintedanib and levulinic acid, preferably approximately equimolar amounts, in isopropanol at an elevated temperature, preferably between about 40° C. and about 60° C. for a suitable time, preferably between about 3 hours and about 10 hours. Preferably, the suspension is allowed to cool to room temperature prior to isolation and drying, if necessary, preferably drying in vacuo and at room temperature.

In another embodiment of the present invention, there is provided a new salt of nintedanib, nintedanib acesulfamate Form APO-I, wherein the molar ratio of nintedanib to acesulfame is approximately 1:1.

Nintedanib acesulfamate Form APO-I can be characterized by a PXRD diffractogram comprising, among other peaks, characteristic peaks, expressed in degrees 2θ (±0.2°), at 12.9° and 14.6°. Preferably, the PXRD diffractogram further comprises at least four peaks, expressed in degrees 2θ (±0.2°), selected from the group consisting of 10.9°, 12.3°, 17.2°, 18.1°, 20.9°, and 21.4°. More preferably, the PXRD diffractogram further comprises peaks, expressed in degrees 2θ (±0.2°), at 10.9°, 12.3°, 17.2°, 18.1°, 20.9°, and 21.4°.

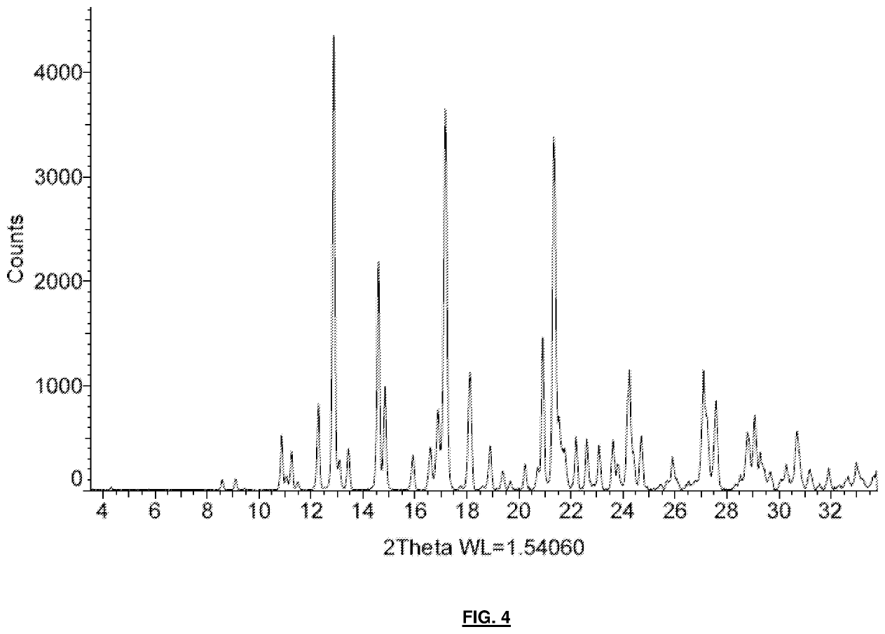

An illustrative PXRD diffractogram of nintedanib acesulfamate Form APO-I, as prepared in Example 4, is shown in FIG. 4. A peak listing, comprising representative peaks from the PXRD diffractogram in FIG. 4, and their relative intensities, is provided in Table 4. Although illustrative of the PXRD diffractogram that is provided for the nintedanib acesulfamate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 4
Relative peak intensities of nintedanib
acesulfamate Form APO-I from FIG. 4

| Angle (2θ) | Relative intensity (%) |
|---|---|
| 10.88 | 12.1 |
| 11.26 | 8.7 |
| 12.29 | 19.0 |
| 12.88 | 100.0 |
| 13.45 | 9.2 |
| 14.59 | 50.5 |
| 14.86 | 22.8 |
| 15.93 | 7.6 |
| 16.60 | 9.5 |
| 16.90 | 17.8 |
| 17.18 | 84.2 |
| 18.12 | 26.1 |
| 18.90 | 9.8 |
| 20.92 | 33.6 |
| 21.35 | 78.0 |
| 24.25 | 26.5 |

As described in Example 4, nintedanib acesulfamate Form APO-I can be prepared by maintaining a suspension of nintedanib and acesulfame, preferably approximately equimolar amounts, in a mixture of isopropanol and water, at an elevated temperature, preferably between about 40° C. and about 60° C. for a suitable time, preferably between about 3 hours and about 10 hours. Preferably, the suspension is allowed to cool to room temperature prior to isolation and drying, if necessary, preferably drying in vacuo and at room temperature.

In another embodiment of the present invention, there is provided a new salt of nintedanib, nintedanib diacesulfamate Form APO-I, wherein the molar ratio of nintedanib to acesulfame is approximately 1:2.

Nintedanib diacesulfamate Form APO-I can be characterized by a PXRD diffractogram comprising, among other peaks, characteristic peaks, expressed in degrees 2θ (±0.2°), at 6.4° and 7.9°. Preferably, the PXRD diffractogram further comprises at least four peaks, expressed in degrees 2θ (±0.2°), selected from the group consisting of 12.0°, 12.8°, 13.3°, 14.6°, 17.1°, and 19.3°. More preferably, the PXRD diffractogram further comprises peaks, expressed in degrees 2θ (±0.2°), at 12.0°, 12.8°, 13.3°, 14.6°, 17.1°, and 19.3°.

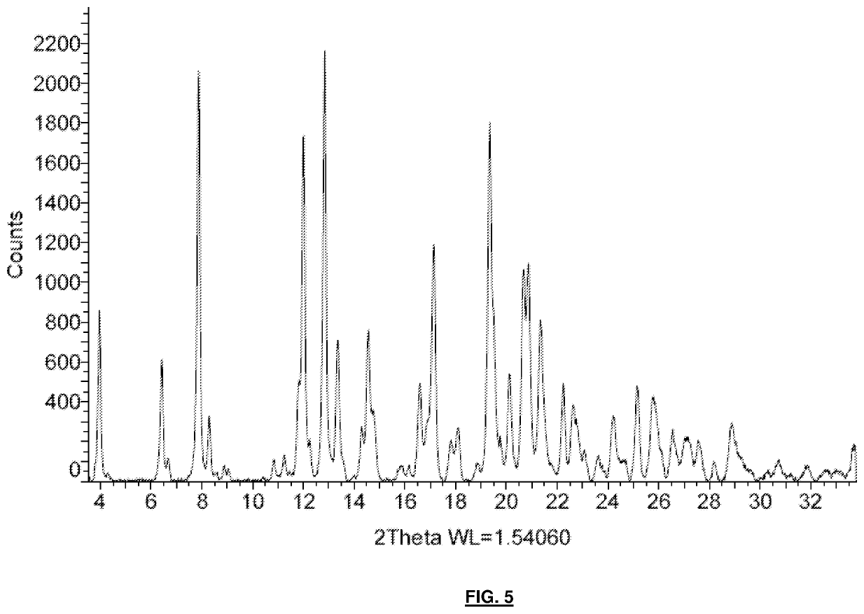

An illustrative PXRD diffractogram of nintedanib diacesulfamate Form APO-I, as prepared in Example 5, is shown in FIG. 5. A peak listing, comprising representative peaks from the PXRD diffractogram in FIG. 5, and their relative intensities, is provided in Table 5. Although illustrative of the PXRD diffractogram that is provided for the nintedanib diacesulfamate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 5
Relative peak intensities of nintedanib
diacesulfamate Form APO-I from FIG. 5

| Angle (2θ) | Relative intensity (%) |
|---|---|
| 3.97 | 39.9 |
| 6.43 | 28.3 |
| 7.88 | 95.4 |
| 8.30 | 15.2 |
| 12.01 | 80.4 |
| 12.84 | 100.0 |
| 13.36 | 32.7 |
| 14.57 | 35.2 |
| 16.59 | 22.7 |
| 17.14 | 55.0 |
| 18.10 | 12.3 |
| 19.35 | 83.4 |
| 20.13 | 24.9 |
| 20.68 | 49.1 |
| 20.87 | 50.7 |

As described in Example 5, nintedanib diacesulfamate Form APO-I can be prepared by maintaining a solution of nintedanib and excess acesulfame, preferably approximately 2 mole equivalents with respect to nintedanib, in methanol at an elevated temperature, preferably between about 40° C. and about 60° C. for a suitable time, preferably between about 3 hours and about 10 hours. Preferably, the suspension is allowed to cool to room temperature prior to isolation and drying, if necessary, preferably drying in vacuo and at room temperature.

In another embodiment of the present invention, there is provided a new salt of nintedanib, nintedanib saccharinate Form APO-I, wherein the molar ratio of nintedanib to saccharin is approximately 1:1.

Nintedanib saccharinate Form APO-I can be characterized by a PXRD diffractogram comprising, among other peaks, characteristic peaks, expressed in degrees 2θ (±0.2°), at 8.1° and 15.7°. Preferably, the PXRD diffractogram further comprises at least four peaks, expressed in degrees 2θ (±0.2°), selected from the group consisting of 7.1°, 8.8°, 10.5°, 12.3°, 17.9°, and 20.5°. More preferably, the PXRD diffractogram further comprises peaks, expressed in degrees 2θ (±0.2°), at 7.1°, 8.8°, 10.5°, 12.3°, 17.9°, and 20.5°.

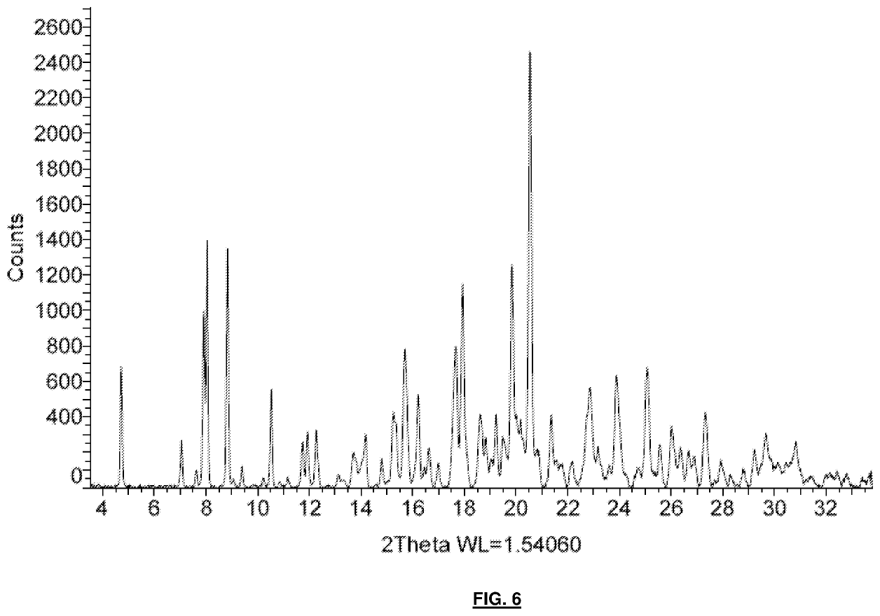

An illustrative PXRD diffractogram of nintedanib saccharinate Form APO-I, as prepared in Example 6, is shown in FIG. 6. A peak listing, comprising representative peaks from the PXRD diffractogram in FIG. 6, and their relative intensities, is provided in Table 6. Although illustrative of the PXRD diffractogram that is provided for the nintedanib saccharinate Form APO-I of the present invention, the relative intensities of the peaks are variable. Thus, depending on a particular sample, the prominence or relative intensity of the peaks observed may differ from those in the illustrative PXRD diffractogram and peak listing.

TABLE 6
Relative peak intensities of nintedanib
saccharinate Form APO-I from FIG. 6

| Angle (2θ) | Relative intensity (%) |
|---|---|
| 4.73 | 27.7 |
| 7.06 | 10.9 |
| 7.92 | 40.4 |
| 8.05 | 56.6 |
| 8.84 | 54.8 |
| 10.53 | 22.5 |
| 11.74 | 10.4 |
| 11.93 | 12.8 |
| 12.28 | 13.2 |
| 13.73 | 8.1 |
| 14.18 | 12.3 |
| 15.28 | 16.0 |
| 15.70 | 31.7 |
| 16.22 | 21.1 |
| 17.67 | 32.2 |
| 17.94 | 46.5 |
| 18.63 | 16.7 |
| 19.23 | 16.6 |
| 19.84 | 50.7 |
| 20.54 | 100.0 |

As described in Example 6, nintedanib saccharinate Form APO-I can be prepared by maintaining a seeded suspension of nintedanib and saccharin, preferably approximately equimolar amounts, in a mixture of isopropanol and water, at an elevated temperature, preferably between about 40° C. and about 60° C. for a suitable time, preferably between about 3 hours and about 10 hours. Preferably, the suspension is allowed to cool to room temperature prior to isolation and drying, if necessary, preferably drying in vacuo and at room temperature.

The suspension may be seeded with nintedanib saccharinate Form APO-II. Preferably, seed crystals can, in the first instance, be prepared by conducting the process for preparation of Form APO-II as described in Example 7. Thereafter, seed crystals for future preparations can also be reserved from Form APO-I prepared, for example, as described in Example 6.

In a further embodiment of the invention, there is provided a pharmaceutical composition comprising nintedanib glutarate, nintedanib hippurate, nintedanib levulinate, nintedanib acesulfamate, nintedanib diacesulfamate, or nintedanib saccharinate, with one or more pharmaceutically acceptable excipients. Preferably, the pharmaceutical composition comprises nintedanib glutarate Form APO-I, nintedanib hippurate Form APO-I, nintedanib levulinate Form APO-I, nintedanib acesulfamate Form APO-I, nintedanib diacesulfamate Form APO-I, or nintedanib saccharinate Form APO-I. Preferably, the pharmaceutical composition is a solid dosage form suitable for oral administration, such as a capsule, soft gelatin capsule, tablet, pill, powder or granulate. Most preferably, the pharmaceutical composition is a tablet or a soft gelatin capsule. Preferably, the pharmaceutical composition provides a dose of nintedanib glutarate, nintedanib hippurate, nintedanib levulinate, nintedanib acesulfamate, nintedanib diacesulfamate, or nintedanib saccharinate that is equivalent to the 100 or 150 mg of nintedanib free base found in OFEV® drug products.

Suitable pharmaceutically acceptable excipients are preferably inert with respect to the nintedanib salts of the present invention and may include, for example, one or more excipients selected from binders such as lactose, starches, modified starches, sugars, gum acacia, gum tragacanth, guar gum, pectin, wax binders, microcrystalline cellulose, methylcellulose, carboxymethylcellulose, hydroxypropyl methylcellulose, hydroxyethyl cellulose, hydroxypropyl cellulose, copolyvidone, gelatine, polyvinylpyrrolidone (PVP), and sodium alginate; fillers or diluents such as lactose, sugar, starches, modified starches, mannitol, sorbitol, inorganic salts, cellulose derivatives (e.g., microcrystalline cellulose, cellulose), calcium sulphate, xylitol, and lactitol; disintegrants such as croscarmellose sodium, crospovidone, polyvinylpyrrolidone, sodium starch glycollate, corn starch, microcrystalline cellulose, hydroxypropyl methylcellulose, and hydroxypropyl cellulose; lubricants such as magnesium stearate, magnesium lauryl stearate, sodium stearyl fumarate, stearic acid, calcium stearate, zinc stearate, potassium benzoate, sodium benzoate, myristic acid, palmitic acid, mineral oil, hydrogenated castor oil, medium-chain triglycerides, poloxamer, polyethylene glycol, and talc; and dispersants or solubility enhancing agents, such as cyclodextrins, glyceryl monostearate, hypromellose, meglumine, Poloxamer, polyoxyethylene castor oil derivatives, polyoxyethylene stearates, polyoxylglycerides, povidone, and stearic acid. Other excipients including preservatives, stabilisers, anti-oxidants, silica flow conditioners, anti-adherents, or glidants may be added as required. Soft gelatin capsule excipients may include, in addition to gelatin, for example, a plasticizer such as glycerin or a polyhydric alcohol such as glycerol; one or more fill excipients selected from carriers such as oily mixtures, polyethylene glycols, glycerides, and fats; and optional ingredients such as surfactants like lecithin. Other suitable excipients and the preparation of solid oral dosage forms are well known to a person of skill in the art, and is described generally, for example, in Remington The Science and Practice of Pharmacy 21^{st} Edition (Lippincott Williams & Wilkins: Philadelphia; 2006; Chapter 45).

Optionally, when the pharmaceutical compositions are solid dosage forms, the solid dosage forms may be prepared with coatings, such as enteric coatings and extended-release coatings, using standard pharmaceutical coatings. Such coatings, and their application, are well known to persons skilled in the art, and are described, for example, in Remington The Science and Practice of Pharmacy 21^{st} Edition (Lippincott Williams & Wilkins: Philadelphia; 2006; Chapter 46).

### EXAMPLES

The following non-limiting examples are illustrative of some of the aspects and embodiments of the invention described herein.

The nintedanib free base used as a starting material in the following examples was prepared by partitioning nintedanib esylate between dichloromethane and aqueous potassium carbonate, followed by layer separation and evaporation of solvent from the organic layer to afford a yellow solid. Other forms of nintedanib free base may be equally suitable as starting material for the examples that follow.

### PXRD Analysis:

PXRD diffractograms were recorded on a Bruker D8 Discover powder X-ray diffractometer (Bruker AXS LLC, Karlsruhe, Germany). The sample holder was oscillated along X and Y axes during the measurement. The generator was a Incoatec Microfocus Source (IμS) Cu tube (λ=1.54060 Å) with a voltage of 50 kV and current of 1.00 mA, using a divergence slit of 0.3 mm and collimator of 0.3 mm. For each sample, one frame was collected using a still scan with a PILATUS3 R 100K-A detector at the distance of 154.72 mm from the sample. Raw data were evaluated using the program DIFFRAC.EVA (Bruker AXS LLC, Karlsruhe, Germany).

### Example 1: Preparation of Nintedanib Glutarate Form APO-I

To nintedanib free base (540 mg) and glutaric acid (140 mg) were added isopropanol (4.4 mL) and water (0.6 mL), and the resulting suspension was stirred at 50° C. for 5 hours. After cooling to room temperature, the solids were collected by vacuum filtration, washed with isopropanol (2×0.7 mL), and dried under vacuum at room temperature for approximately 16 hours. Nintedanib glutarate Form APO-I was obtained as a yellow solid (497 mg, 71% yield). ¹H NMR analysis of the solid (DMSO-d₆) identified a molar ratio of nintedanib:glutaric acid of approximately 1:1. The PXRD diffractogram of a sample prepared by this method is shown in FIG. 1.

¹H-NMR (300 MHz, DMSO-d₆): δ=12.23 (s, 1H), 10.98 (s, 1H), 7.46-7.65 (m, 5H), 7.42 (s, 1H), 7.20 (dd, J=1.5, 8.2 Hz, 1H), 7.13 (d, J=8.5 Hz, 2H), 6.89 (d, J=8.5 Hz, 2H), 5.83 (d, J=8.2 Hz, 1H), 3.77 (s, 3H), 3.06 (br s, 3H), 2.71 (br s, 2H), 2.24 (t, J=7.3 Hz, overlapping br s, 12H), 2.12 (s, 3H), 1.70 (quint, J=7.3 Hz, 2H).

### Example 2: Preparation of Nintedanib Hippurate Form APO-I

To nintedanib free base (540 mg) and hippuric acid (181 mg) was added isopropanol (5 mL) and the resulting suspension was stirred at 50° C. for 5 hours. After cooling to room temperature, the solids were collected by vacuum filtration, washed with isopropanol (3×0.7 mL) and dried under vacuum at room temperature for approximately 16 hours. Nintedanib hippurate Form APO-I was obtained as a yellow solid (523 mg, 73% yield). ¹H NMR analysis of the solid (DMSO-d₆) identified a molar ratio of nintedanib:hippuric acid of approximately 1:1. The PXRD diffractogram of a sample prepared by this method is shown in FIG. 2.

¹H-NMR (300 MHz, DMSO-d₆): δ=12.23 (s, 1H), 10.99 (s, 1H), 8.77 (br t, J=5.5 Hz, 1H), 7.84-7.90 (m, 2H), 7.41-7.65 (m, 9H), 7.20 (dd, J=1.6, 8.2 Hz, 1H), 7.14 (d, J=8.4 Hz, 2H), 6.89 (d, J=8.6 Hz, 2H), 5.83 (d, J=8.2 Hz, 1H), 3.90 (d, J=5.8 Hz, 2H), 3.77 (s, 3H), 3.06 (br s, 3H), 2.71 (br s, 2H), 2.24 (br s, 8H), 2.15 (s, 3H).

### Example 3: Preparation of Nintedanib Levulinate Form APO-I

To nintedanib free base (540 mg) and levulinic acid (131 mg) was added isopropanol (5 mL) and the resulting suspension was stirred at 50° C. for 5 hours. After cooling to room temperature, the solids were collected by vacuum filtration, washed with isopropanol (2×0.7 mL) and dried under vacuum at room temperature for approximately 16 hours. Nintedanib levulinate Form APO-I was obtained as a yellow solid (495 mg, 75% yield). ¹H NMR analysis of the solid (DMSO-d₆) identified a molar ratio of nintedanib:levulinic acid of approximately 1:1. The PXRD diffractogram of a sample prepared by this method is shown in FIG. 3.

¹H-NMR (300 MHz, DMSO-d₆): δ=12.23 (s, 1H), 10.98 (s, 1H), 7.46-7.65 (m, 5H), 7.42 (s, 1H), 7.20 (dd, J=1.5, 8.2 Hz, 1H), 7.13 (d, J=8.5 Hz, 2H), 6.89 (d, J=8.5 Hz, 2H), 5.83 (d, J=8.2 Hz, 1H), 3.77 (s, 3H), 3.07 (br s, 3H), 2.71 (br s, 2H), 2.65 (t, J=6.5 Hz, 2H), 2.37 (t, J=6.5 Hz, 2H), 2.19 (br s, 8H), 2.11 (s, 3H), 2.10 (s, 3H).

### Example 4: Preparation of Nintedanib Acesulfamate Form APO-I

To nintedanib free base (540 mg) and acesulfame (172 mg) were added isopropanol (4.4 mL) and water (0.6 mL) and the resulting suspension was stirred at 50° C. for 5 hours. After cooling to room temperature, the solids were collected by vacuum filtration, washed with isopropanol (3×0.7 mL) and dried under vacuum at room temperature for approximately 16 hours. Nintedanib acesulfamate Form APO-I was obtained as a yellow solid (573 mg, 81% yield). ¹H NMR analysis of the solid (DMSO-d₆) identified a molar ratio of nintedanib:acesulfame of approximately 1:1. The PXRD diffractogram of a sample prepared by this method is shown in FIG. 4.

¹H-NMR (300 MHz, DMSO-d₆): δ=12.25 (s, 1H), 10.99 (s, 1H), 9.26 (br s, 1H), 7.50-7.67 (m, 5H), 7.43 (d, J=1.4 Hz, 1H), 7.20 (dd, J=1.5, 8.2 Hz, 1H), 7.16 (d, J=8.6 Hz, 2H), 6.90 (d, J=8.6 Hz, 2H), 5.83 (d, J=8.2 Hz, 1H), 5.28 (d, J=0.9 Hz, 1H), 3.78 (s, 3H), 3.35 (br s, 2H), 3.07 (br s, 3H), 2.88 (br s, 6H), 2.75 (br s, 3H), 2.45 (br s, 2H), 1.90 (d, J=0.9 Hz, 3H).

### Example 5: Preparation of Nintedanib Diacesulfamate Form APO-I

To nintedanib free base (270 mg) and acesulfame (210 mg) was added methanol (5 mL) and the resulting solution was stirred at 50° C. for 3 hours, during which copious precipitation occurred. After cooling to room temperature, the thick suspension was diluted with ethyl acetate (5 mL), the solids were collected by vacuum filtration, washed with ethyl acetate (2×0.7 mL) and dried under high vacuum at room temperature for approximately 16 hours. Nintedanib diacesulfamate Form APO-I was obtained as a yellow solid (206 mg, 48% yield). ¹H NMR analysis of the solid (DMSO-d₆) identified a molar ratio of nintedanib:acesulfame of approximately 1:2. The PXRD diffractogram of a sample prepared by this method is shown in FIG. 5.

¹H-NMR (300 MHz, DMSO-d₆): δ=12.27 (s, 1H), 11.00 (s, 1H), 9.26 (br s, 1H), 7.50-7.68 (m, 5H), 7.43 (d, J=1.4 Hz, 1H), 7.20 (dd, J=1.5, 8.2 Hz, 1H), 7.17 (d, J=8.6 Hz, 2H), 6.90 (d, J=8.6 Hz, 2H), 5.83 (d, J=8.2 Hz, 1H), 5.56 (d, J=0.9 Hz, 1H), 3.78 (s, 3H), 3.17, 3.09, 2.77 (br singlets overlapping a broad hump, 16H), 2.01 (s, 6H).

### Example 6: Preparation of Nintedanib Saccharinate Form APO-I

To nintedanib free base (540 mg) and saccharin (183 mg) were added isopropanol (4.4 mL) and water (0.6 mL) and the resulting suspension was stirred at 50° C. for 3 hours, at which all solids dissolved. After cooling to room temperature, the solution was seeded with nintedanib saccharinate Form APO-II and copious precipitation occurred. Water (1.5 mL) was added, and the resulting suspension was stirred at room temperature for 72 hours. The solids were collected by vacuum filtration, washed with isopropanol (1×0.7 mL), and dried under vacuum at room temperature for approximately 16 hours. Nintedanib saccharinate Form APO-I was obtained as a yellow solid (235 mg, 32% yield). ¹H NMR analysis of the solid (DMSO-d₆) identified a molar ratio of nintedanib:saccharin of approximately 1:1. The PXRD diffractogram of a sample prepared by this method is shown in FIG. 6.

¹H-NMR (300 MHz, DMSO-d₆): δ=12.25 (s, 1H), 10.99 (s, 1H), 9.24 (br s, 1H), 7.50-7.67 (m, 9H), 7.42 (d, J=1.3 Hz, 1H), 7.20 (dd, J=1.5, 8.2 Hz, 1H), 7.16 (d, J=8.6 Hz, 2H), 6.90 (d, J=8.6 Hz, 2H), 5.83 (d, J=8.2 Hz, 1H), 3.77 (s, 3H), 3.07 (br s, 5H), 2.89 (br s, 4H), 2.73 (br s, 5H), 2.50 (br s, 2H).

### Example 7: Preparation of Nintedanib Saccharinate Form APO-II

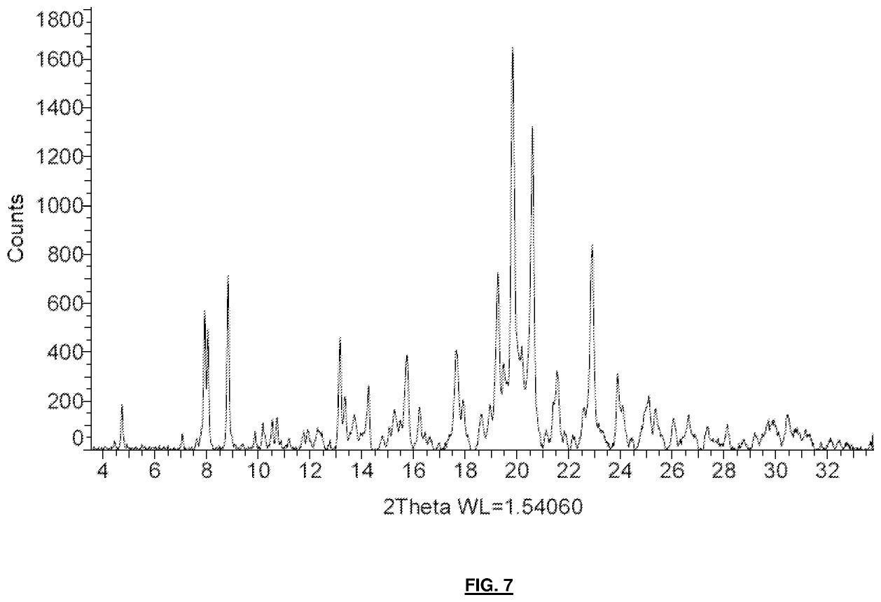

To nintedanib free base (540 mg) and saccharin (188 mg) was added isopropanol (5 mL) and the resulting suspension was stirred at 50° C. for 5 hours. After cooling to room temperature, the solids were collected by vacuum filtration, washed with isopropanol (3×0.7 mL), and dried under vacuum at room temperature for approximately 16 hours. Nintedanib saccharinate Form APO-II was obtained as a yellow solid (600 mg, 80% yield). ¹H NMR analysis of the solid (DMSO-d₆) identified a molar ratio of nintedanib:saccharin:isopropranol of approximately 1:1:0.5. The PXRD diffractogram of a sample prepared by this method is shown in FIG. 7.

¹H-NMR (300 MHz, DMSO-d₆): δ=12.25 (s, 1H), 10.99 (s, 1H), 9.28 (br s, 1H), 7.50-7.68 (m, 9H), 7.43 (d, J=1.2 Hz, 1H), 7.20 (dd, J=1.5, 8.2 Hz, 1H), 7.16 (d, J=8.4 Hz, 2H), 6.90 (d, J=8.5 Hz, 2H), 5.83 (d, J=8.2 Hz, 1H), 4.35 (br s, 0.5H), 3.78 (s, overlapping isopropanol septet, 3.5H), 3.07 (br s, 5H), 2.90 (br s, 4H), 2.73 (br s, 5H), 2.51 (br s, 2H), 1.04 (d, J=6.1 Hz, 3H).

### Example 8: Stability Testing

Samples were kept in an uncovered vial in a stability chamber maintained at 40° C./75% relative humidity (RH) for the specified period prior to PXRD analysis to evaluate form stability. In addition to PXRD analysis, ¹H NMR spectroscopy of nintedanib hippurate Form APO-I following storage showed no change. Results are provided in Table 8.

TABLE 8
Form Stability of Nintedanib Salt Forms at High
Temperature and Humidity

| Form | Time (months) | Result |
|---|---|---|
| Nintedanib glutarate Form APO-I | 1 | NC |
| Nintedanib hippurate Form APO-I | 3 | NC |
| Nintedanib levulinate Form APO-I | 3 | NC |
| Nintedanib acesulfamate Form APO-I | 1 | NC |
| Nintedanib diacesulfamate Form APO-I | 3 | NC |
| Nintedanib saccharinate Form APO-I | 1 | NC |

NC: no change in PXRD

1. A glutarate salt of nintedanib.

2. The glutarate salt of nintedanib of claim 1, wherein the molar ratio of nintedanib to glutaric acid is approximately 1:1.

3. The glutarate salt of claim 2, characterized by a PXRD diffractogram comprising peaks, expressed in degrees 2θ (±0.2°), at 10.1° and 12.9°.

4. The glutarate salt of claim 3, further comprising at least four peaks in the PXRD diffractogram, expressed in degrees 2θ (±0.2°), selected from the group consisting of: 11.0°, 15.1°, 17.4°, 18.0°, 19.5°, and 20.2°.

5. A hippurate salt of nintedanib.

6. The hippurate salt of nintedanib of claim 5, wherein the molar ratio of nintedanib to hippuric acid is approximately 1:1.

7. The hippurate salt of claim 6, characterized by a PXRD diffractogram comprising peaks, expressed in degrees 2θ (±0.2°), at 9.1° and 9.8°.

8. The hippurate salt of claim 7, further comprising at least four peaks in the PXRD diffractogram, expressed in degrees 2θ (±0.2°), selected from the group consisting of: 10.1°, 10.6°, 13.0°, 13.3°, 14.6°, and 20.9°.

9. A levulinate salt of nintedanib.

10. The levulinate salt of nintedanib of claim 9, wherein the molar ratio of nintedanib to levulinic acid is approximately 1:1.

11. The levulinate salt of claim 10, characterized by a PXRD diffractogram comprising peaks, expressed in degrees 2θ (±0.2°), at 8.8° and 10.9°.

12. The levulinate salt of claim 11, further comprising at least four peaks in the PXRD diffractogram, expressed in degrees 2θ (±0.2°), selected from the group consisting of: 8.1°, 11.6°, 12.1°, 16.3°, 17.6°, and 20.5°.

13. An acesulfamate salt of nintedanib.

14. The acesulfamate salt of claim 13, wherein the molar ratio of nintedanib to acesulfame is approximately 1:1, and the salt is characterized by a PXRD diffractogram comprising peaks, expressed in degrees 2θ (±0.2°), at 12.9° and 14.6°.

15. The acesulfamate salt of claim 14, further comprising at least four peaks in the PXRD diffractogram, expressed in degrees 2θ (±0.2°), selected from the group consisting of: 10.9°, 12.3°, 17.2°, 18.1°, 20.9°, and 21.4°.

16. The acesulfamate salt of claim 13, wherein the molar ratio of nintedanib to acesulfame is approximately 1:2, and the salt is characterized by a PXRD diffractogram comprising peaks, expressed in degrees 2θ (±0.2°), at 6.4° and 7.9°.

17. The acesulfamate salt of claim 16, further comprising at least four peaks in the PXRD diffractogram, expressed in degrees 2θ (±0.2°), selected from the group consisting of: 12.0°, 12.8°, 13.3°, 14.6°, 17.1°, and 19.3°.

18. A saccharinate salt of nintedanib, wherein the molar ratio of nintedanib to saccharin is approximately 1:1, that is characterized by a PXRD diffractogram comprising peaks, expressed in degrees 2θ (±0.2°), at 8.1° and 15.7°.

19. The saccharinate salt of claim 19, further comprising at least four peaks in the PXRD diffractogram, expressed in degrees 2θ (±0.2°), selected from the group consisting of: 7.1°, 8.8°, 10.5°, 12.3°, 17.9°, and 20.5°.

20. A pharmaceutical composition comprising the salt of nintedanib according to claim 1, and one or more pharmaceutically acceptable excipients.
